(19) United States
(12) Patent Application Publication
(10) Pub. No.: US 2013/0046363 A1
(43) Pub. Date: Feb. 21, 2013

(54) Laser Therapy Device and Method for Use

(71) Applicant: Michael K. Thomas, Henderson, MN (US)

(72) Inventor: Michael K. Thomas, Henderson, MN (US)

(21) Appl. No.: 13/589,768

(22) Filed: Aug. 20, 2012

Related U.S. Application Data
(60) Provisional application No. 61/525,280, filed on Aug. 19, 2011.

(51) Int. Cl. A61N 5/067

(52) U.S. Cl. 607/89

(57) ABSTRACT

A laser therapy device and method of treatment are disclosed. The laser therapy device includes a housing, laser source, power source, vial, and vial holder. Laser light will shine through the vial containing an aqueous solution where the laser will acquire the signature of the aqueous fluid. A method for the treatment of an ailment where laser light is transmitted through a solution and transmitted to a target source.

### CROSS REFERENCE TO RELATED APPLICATIONS

This application claims the benefit of U.S. Provisional Patent Application Ser. No. 61/525,280 filed 2001 Aug. 19 to the above named inventor.

### FEDERALLY SPONSORED RESEARCH OR DEVELOPMENT Not Applicable SEQUENCE LISTING, A TABLE, OR A COMPUTER PROGRAM

Not Applicable

### FIELD OF THE INVENTION

The present invention relates to a low level laser therapy device and method for treatment using a low level laser emitting device and a lens containing an aqueous botanical, herbal, or amino-acid solution.

### BACKGROUND OF THE INVENTION

Low levels of laser energy have been known to have a non-thermal, biostimulative effect on biological tissues. The therapeutic application of these low levels of laser energy has been known as low level laser therapy (“LLLT”). LLLT has been known to create beneficial clinical results in the treatment of various musculoskeletal, neurological, soft tissue, and chronic pain conditions. LLLT is non-invasive and delivers photons to tissue inciting photochemical results at a cellular level.

In addition to LLLT, it is known that the topical treatment of botanicals, herbs, and certain amino acid compounds may also deliver similar therapeutic properties. It is also known that, laser light is capable of carrying the harmonic or vibrational frequencies of any liquid through which it passes. Currently there is no device that utilizes the therapeutic benefits of both laser light and the harmonic or vibrational frequency of an aqueous herbal, aqueous botanical, or aqueous amino acid solution.

Therefore, there exists a need for a laser emitting device that can safely and effectively transmit laser light through a prepared solution for therapeutic use.

### SUMMARY OF THE INVENTION

A Laser therapy device according to the present invention includes a housing enclosing a laser source capable of emitting low level laser light through an aperture in the housing. Preferably, the laser source emits laser light at a power level within the range of 200 mW (milliwatts) to 500 mW. The laser source operates in the visible spectrum range with a wavelength corresponding to the colors of Red, at a wavelength between 635 nm (nanometers) and 650 nm, Green, at an approximate wavelength of 535 nm, Orange, Yellow, White, Blue, at an approximate wavelength of 445 nm, or Violet, at an approximate wavelength of 405 nm.

The housing may include one laser source for a given color or multiple laser sources for each given color, whereby the housing may contain up to seven selectable laser sources representing a wavelength corresponding to the colors of Red, Green, Orange, Yellow, White, Blue, or Violet.

The housing includes a power source, a power switch, a power indicator, a key switch, a power key, a remote connector key, an aperture locking mechanism, an aperture focusing mechanism, and a vial holding cap receiver. The power source provides power to the device and may be composed of a replaceable or rechargeable battery source providing direct current to the device. The power switch, will allow a user to power on the device or to power off the device. The power switch may be a switch, a button, a toggle, or similar components allowing a user easily turn the device on or off. The power indicator will indicate if the device is turned on. The power indicator may be an illuminated light, light emitting diode, or similar component.

The key switch allows a user to disable or lock the device using the power key to prevent inadvertent operation by disabling the power switch. The remote connector key is a further safety feature preventing inadvertent usage of device. The remote connector key is in removable communication with a port located on housing. The remote connector, when inserted into the port allows for proper operation of the device. When the remote connector key is removed from the device, the device will not operate. The remote connector key and the key switch create added security by requiring a user to have both the power key and the remote connector key properly engaged to operate the device.

The aperture locking mechanism allows a user to close or open the aperture in the housing. The aperture focusing mechanism allows a user to alter the size of the aperture located in the housing, whereby a change in the size of the aperture focuses the laser light to a given target area.

A vial holding cap is in removable communication with the housing and includes a laser aperture, a vial aperture, and an attachment means. The vial holding cap is received in the housing via the attachment means. This receipt may be threaded, whereby the vial holding cap receiver is threaded to securely receive the vial holding cap through the threaded receipt of the attachment means. Although a threaded assembly is the preferred connection between the vial holding cap and the housing, other forms of assembly may be used, such as a snap-fit, a friction-fit, a magnetic fit, or other similar attachment.

The laser aperture is aligned with the laser source and allows the laser beam to exit the housing and onto the treatment surface. The vial aperture intersects the laser aperture and is sized to removably receive a vial. The vial is filled with an aqueous solution and sealed to prevent degradation. The vial is transparent to allow the laser light to travel through the vial and the enclosed aqueous solution.

The aqueous solution in the vial may be composed of, but not limited to, the following botanical extracts and amino acid compounds: Aloe Vera, Ajos Sacha, Amor Seco, Anamu, Andiroba, Ayapana, Bellaco-Caspi, Bobinsana, Boldo, Catuaba, Chanca Piedra, Chuchuhuasi, Copaiba, Cumaseba, Embauba, Erva tostau, Gervau, Graviola, Guacatonga, Huacapu, Iporuru, Jatoba, Jergon Sacha, Juazeiro, Jerubeba, Kalanchoe, Manaca, Matico, Mulateiro, Mulungu, Mutamba, Samambala, Simarouba, Tamamuri, Tayuya, Ubos, Una de Gato, Chaparral, Huperzine A, L-Arginine, L-theanine, MSM, Siberian Golden Root, Vinpocetine. Several formulations of the above listed ingredients may be used to create a system of multiple vials for the treatment for common ailments.

In the preferred embodiment, the system of multiple vials formulated for specific ailments includes a vial formulated for the reduction/elimination of pain, a vial formulated for the treatment of chronic pain, a vial formulated for the treatment of pain related to inflammation, a vial formulated for the treatment of deep tissue and chronic pain, and a vial formulated pain reduction with a combination of cellular energizing. The vials for the treatment of various ailments may be specifically identified by a classification system for the ailment to be treated. This classification system may be based on colors, letters, numbers, or a specific identification of the ailment.

In the preferred embodiment of the present invention, the laser therapy device and the vial are used to treat a patient's ailment by passing laser light from the device through the vial and onto a treatment area. Accordingly, a method for the use of this system is described.

According to this method, a treatment provider will first identify the ailment to be treated. After identifying the ailment, the treatment provider will then select the proper treatment vial and laser source color. The treatment provider will then select the proper housing containing the appropriate laser source, place the appropriate vial into the vial aperture, power on the device, and administer the laser light traveling through the vial and the laser aperture to the target area for a specified period of time.

According to this method, a treatment plan is created for a patient's ailments whereby a practitioner will administer a series of laser treatments consisting of a specified laser color and vial over a period of time. This treatment plan may alternate in both the aqueous solution type contained in the vial and the color of the laser used.

### BRIEF DESCRIPTION OF THE SEVERAL VIEWS OF THE DRAWING(S)

The accompanying drawings are included to provide a further understanding of the present invention and are incorporated in and constitute a part of this specification. The drawings illustrate exemplary embodiments of the present invention and together with the description serve to further explain the principles of the invention. Other aspects of the invention and the advantages of the invention will be better appreciated as they become better understood by reference to the Detailed Description when considered in conjunction with accompanying drawings, and wherein:

FIG. 1 is a perspective view of the laser therapy device, according to the present invention;

### DETAILED DESCRIPTION OF THE INVENTION

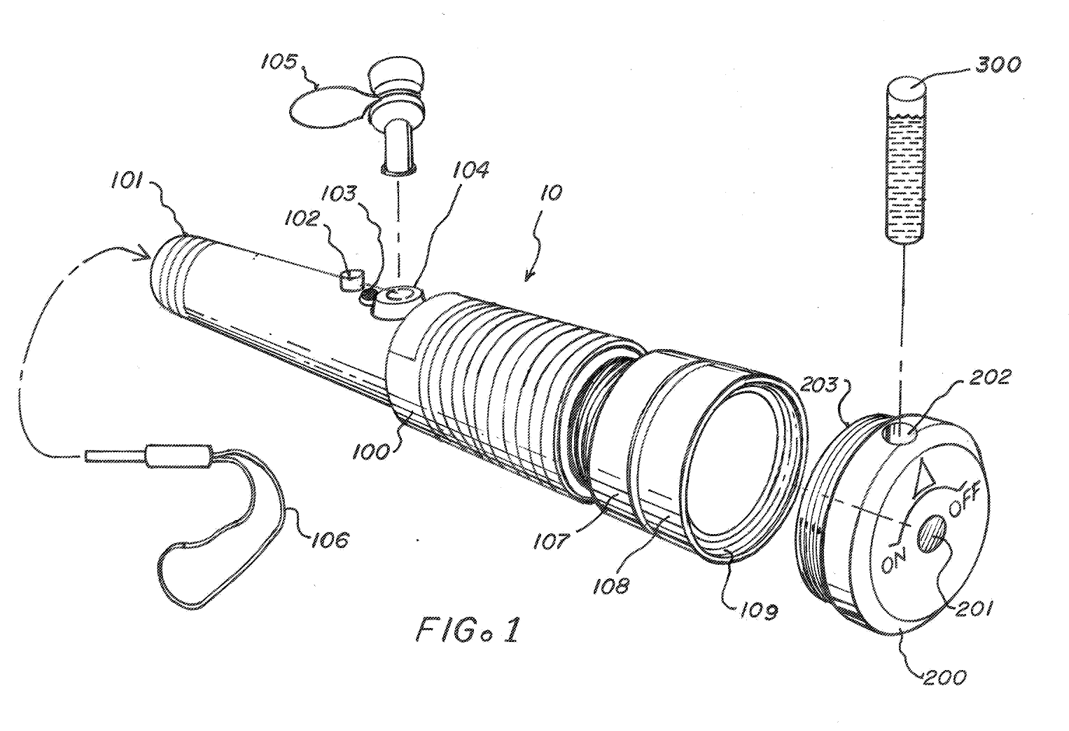

Referring now to FIG. 1, of a laser therapy device according to the present invention, generally referred to as 10. The laser therapy device 10 includes a housing 100. The housing 100 includes a laser source capable of emitting low level laser light at a power level within the range of 200 mW (milliwatts) to 500 mW through an aperture (not pictured) in the housing 100. In the preferred embodiment of the present invention, the laser source will be operating in the visible spectrum range with a wavelength corresponding to the colors of Red, at a wavelength between 635 nm (nanometers) and 650 nm, Green, at an approximate wavelength of 535 nm, Orange, Yellow, White, Blue, at an approximate wavelength of 445 nm, or Violet, at an approximate wavelength of 405 nm. The housing 100 may include one laser source for a given color or multiple laser sources for each given color, whereby the housing 100 may contain up to seven selectable laser sources representing a wavelength corresponding to the colors of Red, Green, Orange, Yellow, White, Blue, or Violet.

The housing 100 includes a power source 101, a power switch 102, a power indicator 103, a key switch 104, a power key 105, a remote connector key 106, a aperture locking mechanism 107, a aperture focusing mechanism 108, and a vial holding cap receiver 109. The power source 101 provides power to the device 10 and may be composed of a replaceable or rechargeable battery source providing direct current to the device. The power switch 102, will allow a user to power on the device or power off the device. The power switch 102, may be a switch, a button, a toggle, or a similar component allowing a user easily turn the device on or off. The power indicator 103 will indicate if the device is turned on. The indicator 103 may be an illuminated light, light emitting diode, or similar component.

The key switch 104 allows a user to disable or lock the device using the power key 105 to prevent inadvertent operation by disabling the power switch 102. The remote connector key 106 is a further safety feature preventing inadvertent usage of the device 10. The remote connector key 106 is in removable communication with a port located on the housing 100. The remote connector 106 when inserted into the port allows for the proper operation of the device, when the remote connector key 106 is removed from the device 10, the device will not operate. The remote connector key 106 and the key switch 104 create added security by requiring a user to have both the power key 105 and the remote connector key 106 to properly operate the device 10.

The aperture locking mechanism 107 allows a user to close or open the aperture in the housing 100. An aperture focusing mechanism 108 allows a user to alter the size of the aperture located in the housing 100, whereby a change in the size of the aperture focuses the laser to a given target area.

A vial holding cap 200 is in removable communication with the housing 100 and includes a laser aperture 201, a vial aperture 202, and an attachment means 203. The vial holding cap 200 is received in the housing 100 via the attachment means 203. This receipt may be threaded, whereby the vial holding cap receiver 109 is threaded to securely receive the vial holding cap 200 through the threaded receipt of attachment means 203. Although a threaded assembly is the preferred connection between the vial holding cap 200 and the housing 100, other forms of assembly may be used, such as snap-fit, friction-fit, magnetic fit, or other similar attachment.

The laser aperture 201 is aligned with the laser source and allows the laser beam to exit the housing and onto the treatment surface. The vial aperture 202 intersects the laser aperture 201 and is sized to removably receive a vial 300. The vial 300 is filled with an aqueous solution and sealed to prevent degradation. The vial 300 is transparent allowing the laser beam to travel through the vial 300 and the enclosed aqueous solution.

The aqueous solution in the vial 300 may be composed of, but not limited to, the following botanical extracts and amino acid compounds: Aloe Vera, Ajos Sacha, Amor Seco, Anamu, Andiroba, Ayapana, Bellaco-Caspi, Bobinsana, Boldo, Catuaba, Chanca Piedra, Chuchuhuasi, Copaiba, Cumaseba, Embauba, Erva tostau, Gervau, Graviola, Guacatonga, Huacapu, Iporuru, Jatoba, Jergon Sacha, Juazeiro, Jerubeba, Kalanchoe, Manaca, Matico, Mulateiro, Mutamba, Samambala, Simarouba, Tamamuri, Tayuya, Ubos, Una de Gato, Chaparral, Huperzine A, L-Arginine, L-lysine, L-theanine, MSM, Siberian Golden Root, Vinpocetine. Several formulations of the above listed ingredients may be used to create a system of multiple vials 300 for the treatment for common ailments.

In the preferred embodiment, the system of multiple vials 300 formulated for specific ailments includes a vial formulated for the reduction/elimination of pain, a vial formulated for the treatment of chronic pain, a vial formulated for the treatment of pain related to inflammation, a vial formulated for the treatment of deep tissue and chronic pain, and a vial formulated pain reduction with a combination of cellular energizing. These vials 300 for the treatment of ailments may be specifically identified by a classification system for the ailment to be treated. This classification system may be based on colors, letters, numbers, or a specific identification of the ailment.

In the preferred embodiment of the present invention, the laser therapy device 10 and the vial 300 are used to treat a patient's ailment by passing laser light from the device 10 through the vial 300 and onto the treatment area. Accordingly, a method for the use of this system is described.

According to this method, a treatment provider will first identify the ailment to be treated. After identifying the ailment, the treatment provider will then select the proper treatment vial 300 and laser source color. The treatment provider will then select the proper housing 100 containing the appropriate laser source, place the appropriate vial 300 into the vial aperture 202, power on device 10, and administer the laser light traveling through the vial 300 and the laser aperture 201 to the target area for a specified period of time.

According to this method, a treatment plan is created for a patient's ailments whereby a practitioner will administer a series of laser treatments consisting of a specified laser color and vial over a period of time. This treatment plan may alternate in both the aqueous solution type contained in the vial and the color of the laser used.

What is claimed is:

1. A device for administering laser therapy, the device comprising:
a laser source, the laser source capable of emitting a low level of laser light;
a power source, the power source providing power for the laser source;
a housing, the housing enclosing the laser source and the power source, the housing including:
a vial holding cap, the vial holding cap comprising:
a laser aperture aligned with the laser source, the laser aperture allowing laser light to exit the housing;
a vial aperture, the vial aperture intersecting the laser aperture and sized to receive a vial; and
the vial sized to be received in the vial aperture, the vial being transparent and containing an aqueous solution, wherein the laser light emitted from the laser source travels through the vial.

2. A device as in claim 1, wherein the laser source operates at a power level within a range of 200 to 500 milliwatts.

3. A device as in claim 1, wherein the laser source is colored.

4. A device as in claim 1, wherein the laser source is red, green, orange, yellow, white, blue, or violet.

5. A device as in claim 1, wherein the aqueous solution in the vial is composed of Aloe Vera, Ajos Sacha, Amor Seco, Anamu, Andiroba, Ayapana, Bellaco-Caspi, Bobinsana, Boldo, Catuaba, Chanca Piedra, Chuchuhuasi, Copaiba, Cumaseba, Embauba, Erva tostau, Gervau, Graviola, Guacatonga, Huacapu, Iporuru, Jatoba, Jergon Sacha, Juazeiro, Jerubeba, Kalanchoe, Manaca, Matico, Mulateiro, Mulungu, Mutamba, Samambala, Simarouba, Tamamuri, Tayuya, Ubos, Una de Gato, Chaparral, Huperzine A L-Arginine, L-lysine, L-theanine, MSM, Siberian Golden Root, or Vinpocetine.

6. A housing for a laser therapy device, the device having a laser source capable of emitting a low level of laser light and having a power source, the housing comprising:
a vial holding cap, the vial holding cap attached to the housing and the vial holding cap comprising:
a laser aperture aligned with the laser source, the laser aperture allowing laser light to exit the housing;
a vial aperture, the vial aperture intersecting the laser aperture and sized to receive a vial; and
the vial sized to be received in the vial aperture, the vial being transparent and containing an aqueous solution, wherein the laser light emitted from the laser source travels through the vial.

7. A device as in claim 6, wherein the housing comprises:
a power switch, the power switch initiating power to the device;
a power indicator, the power indicator alerting a user that the device is powered on;
a key switch;
a remote connector key; and
a aperture locking mechanism.

8. A device as in claim 6, wherein the laser source operates at a power level within a range of 200 to 500 milliwatts.

9. A device as in claim 6, wherein the laser source is colored.

10. A device as in claim 6, wherein the laser source is red, green, orange, yellow, white, blue, or violet.

11. A device as in claim 6, wherein the aqueous solution in the vial is composed of Aloe Vera, Ajos Sacha, Amor Seco, Anamu, Andiroba, Ayapana, Bellaco-Caspi, Bobinsana, Boldo, Catuaba, Chanca. Piedra, Chuchuhuasi, Copaiba, Cumaseba, Embauba, Erva tostau, Gervau, Graviola, Guacatonga, Huacapu, Iporuru, Jatoba, Jergon Sacha, Juazeiro, Jerubeba, Kalanchoe, Manaca, Matico, Mulateiro, Mulungu, Mutamba, Samambala, Simarouba, Tamamuri, Tayuya, Ubos, Una de Gato, Chaparral, Huperzine A, L-Arginine, L-lysine, L-theanine, MSM, Siberian Golden Root, or Vinpocetine.

12. A laser therapy method for the surface treatment of ailments, the method comprising:
selecting a transparent vial containing an aqueous solution;
aligning a laser source with the vial such that laser light shines through the vial; and
applying the laser light emanating through the vial to the ailment surface.

13. The method as in claim 12, wherein the laser source operates at a power level within a range of 200 to 500 milliwatts.

14. The method as in claim 12, wherein the laser source is colored.

15. The method as in claim 12, wherein the laser source is red, green, orange, yellow, white, blue, or violet.

16. The method as in claim 12, wherein the aqueous solution in the vial is composed of Aloe Vera, Ajos Sacha, Amor Seco, Anamu Andiroba, Ayapana, Bellaco-Caspi, Bobinsana, Boldo, Catuaba, Chanca Piedra, Chuchuhuasi, Copaiba, Cumaseba, Embauba, Erva tostau, Gervau, Graviola, Guacatonga, Huacapu, Iporuru Jatoba, Jergon Sacha, Juazeiro, Jerubeba, Kalanchoe, Manaca, Matico, Mulateiro, Mulungu, Mutamba, Samambala, Simarouba, Tamamuri, Tayuya, Ubos, Una de Gato, Chaparral, Huperzine A, L-Arginine, L-lysine, L-theanine, MSM, Siberian Golden Root, or Vinpocetine.
